(19) United States
(12) Patent Application Publication
(10) Pub. No.: US 2021/0388455 A1
(43) Pub. Date: Dec. 16, 2021

(54) Kobophenol A for the treatment of Corona Virus 2 (SARS-CoV-2) infection

(71) Applicants: Suresh Gangadevi, Balayapalli Mandal (IN); Yin Na, Bengbu (CN); Wang Ke, Bengbu (CN); Vishnu Nayak Badavath, Indalwai Mandal (IN)

(72) Inventors: Suresh Gangadevi, Balayapalli Mandal (IN); Yin Na, Bengbu (CN); Wang Ke, Bengbu (CN); Vishnu Nayak Badavath, Indalwai Mandal (IN)

(21) Appl. No.: 17/347,261

(22) Filed: Jun. 14, 2021

(30) Foreign Application Priority Data
Jun. 13, 2020 (IN) 202041024910

(51) Int. Cl. C12Q 1/70

(52) U.S. Cl. CPC C12Q 1/701

(57) ABSTRACT

The present disclosure relates to an in-silico analysis for inhibitors of Severe Acute Respiratory Syndrome Corona Virus 2 (SARS-CoV-2). The in-silico screening of Kobophenol A confirmed the effective binding at two positions, firstly at ACE2/Spike interface and secondly at the hydrophobic pocket by destabilizing the complex formation. Kobophenol A inhibited the cell death caused by viral infection without inducing cell toxicity in the absence of viral infection. The molecular dynamics of Kobophenol A indicated the stability of binding of Kobophenol A through hydrogen bond and stabilized at Y495 and K353 with an average distance of 2.95 Å. The binding affinity of Kobophenol A to the ACE2/Spike interface region and the ACE2 hydrophobic pocket is computed to be −19.0±4.3 and −24.9±6.9 kcal/mol respectively. Kobophenol A is useful as a potential drug for treatment of COVID-19.

### CROSS-REFERENCE TO RELATED APPLICATION

This application claims priority to and the benefit of Indian Patent Application No. 202041024910, filed Jun. 13, 2020, the entire contents of which are incorporated herein by reference.

### TECHNICAL FIELD OF THE DISCLOSURE

The present disclosure relates to in silico drug design analysis to examine the natural-based compounds as inhibitors of Severe Acute Respiratory Syndrome Corona Virus 2 (SARS-CoV-2). More particularly, the disclosure relates to the analysis of Kobophenol A based on the interaction between the human Angiotensin-Converting Enzyme 2 (ACE2) receptor and Spike receptor-binding domain (S1-RBD) of SARS-CoV-2 for treatment of infection caused by Corona Virus Disease-2019 (Covid-19).

### BACKGROUND

Severe Acute Respiratory Syndrome Corona Virus 2 (SARS-CoV-2), commonly called coronavirus, is an enveloped positive-sense Ribose Nucleic Acid (RNA) virus known as a human and animal pathogen with a large RNA genome. Coronaviruses are the largest group of viruses belonging to the Nidovirales order including Coronaviridae, Arteriviridae, Mesoniviridae and Roniviridae families They all contain very large genomes for RNA viruses, with some viruses having the largest identified RNA genomes containing up to 33.5 kilobase (kb) genomes.

Coronavirus particles contain four main structural proteins, namely spike (S), membrane (M), envelope (E) and nucleocapsid (N) proteins and are encoded within the 3′ end of the viral genome.

SARS-CoV-2 causes an infectious disease called COVID-19 mainly transmitted through droplets generated when an infected person coughs, sneezes or exhales. COVID-19, first detected in Wuhan, China, is characterized by flu like symptoms and pneumonia primarily affecting the lungs.

COVID-19 is characterized by the development of mild to moderate illness in patients who may recover without hospitalization. The most common symptoms are fever, dry cough, and tiredness. More severe cases of the disease results in difficulty in breathing, shortness of breath, chest pain and sometimes even death.

Coronavirus infection is caused by replication of a virus in the host cell. Viral replication is the formation of biological viruses during the infection process in the target host cells. The virus is incapable of self-replication and will only multiply in an internal cellular environment. Viruses lack subcellular organelles such as nuclei, mitochondria, ribosomes as well as cytoplasmic components that are necessary for the synthesis of their own structural components such as nucleic acids, proteins, carbohydrates, and lipids. Viral replication is a complex process involving different mechanisms of the host cell for replication including signaling molecules and signal transduction processes. Although the replicative life cycle of viruses differs between species and category of virus, there are six basic stages that are essential for viral replication, namely adhesion, penetration, uncoating, replication, assembly, and virion release.

Scientists around the world are trying different therapies or medications and even repurposing existing drugs to combat the viral spread. A leprosy drug sepsivac, which comprises an immunomodulator and, according to the Indian Council of Medical Research's findings, helps to treat and even reduce the mortality rate in critically ill patients, has been repurposed for use against COVID-19.

Similarly, hydroxychlorquine (HCQ) has been one of the controversial drugs in the news of late. The anti-malarial drug also used to treat certain auto-immune diseases and arthritis conditions has shown immense promise in treating some of the symptoms associated with coronavirus. Even though studies are still underway, warnings have been issued against widescale use of the HCQ drugs and in some places has been reserved for use only in hospital settings and to be administered to frontline workers.

In addition, the combination of lopinavir and ritonavir has also shown promising results in reducing the severity of the symptoms in patients with COVID-19. The combination may be effective in preventing the adhesion of virus to host cell and reproduction in the host immune system.

Kobophenol A is a natural oligomeric stilbenoid isolated from Caragana genus and is a tetramer of resveratrol. Kobophenol A is known to inhibit acetylcholinesterase activity and exhibit neuroprotective and cardioprotective activities. However, the analysis of X-ray binding studies is useful in determination of free binding affinity of Kobophenol A to determine its inhibitory activity.

The development of new drugs is a lengthy process and involves multiple factors. The use of the natural compounds that possess tremendous structural range and unique chemical diversity serve as excellent starting points for inspiring new drug discovery. With the development in the current technological approaches, natural compounds remain potentially transformative drugs for many health conditions. The growing understanding of efficient antiviral drug development has led to the exploration of natural compounds as an important approach for identifying effective COVID-19 treatments.

To date, there have been very few in silico attempts to find small molecule inhibitors of the interaction between ACE2 and spike S1-RBD.

Most of these drugs including chloroquine, hydroxychloroquine, remdesivir, favipiravir and the recently known EIDD-2801 are in clinical trials. However, no specific vaccine has been developed to treat COVID-19.

The Patent Application No. KR20090011458A entitled “The composition containing kobophenol A, having prevention and treatment effects for neuronal diseases” discloses a composition for the prevention and treatment of cerebral neurological diseases containing kobophenol A as an active ingredient, and more specifically, bone tobacco kobophenol A. The composition has an inhibitory effect on neuronal cell death and is effective for diseases caused by neuronal cell death, such as neurodegenerative diseases such as Alzheimer's disease and Parkinson's disease. The disclosure relates to a new use of kobophenol A, which can be used as a material for medicines and health foods.

The Patent Application No. KR20200026550A entitled “An antiviral composition comprising extract of caragana sinica or compound derived from the same as an active ingredient” discloses antiviral composition comprising the extract of gingival limb or a compound derived therefrom as an active ingredient and having a neuraminidase inhibitory activity of an influenza virus.

The publication entitled “Kobophenol A Isolated from Roots of Caragana sinica (Buc'hoz) Rehder Exhibits Anti-inflammatory Activity by Regulating NF-κB Nuclear Translocation in J774A.1 Cells” by Hana Cho et. al. discloses that KPA treatment significantly suppressed the production of nitric oxide (NO) by inhibiting inducible nitric oxide synthase (iNOS) expression in a dose-dependent manner without cytotoxicity. KPA also inhibited pro-inflammatory cytokine gene expression and production such as interleukin-1β (IL-1β) and interleukin-6 (IL-6) in LPS-stimulated J774 A.1 cells. As continuing study on the mechanisms involved, the study also confirmed that these effects of KPA were related to the inhibition of nuclear factor-κB (NF-κB) pathway including the suppression of IκB kinase α/β (IKKα/β) phosphorylation and translocation of NF-κB into the nucleus. The study is the first to demonstrate that KPA isolated from C. sinica suppresses the expression of inflammatory mediators and cytokines by inhibiting NF-κB nuclear translocation in LPS-stimulated J774 A.1 macrophages. KPA may be a potential candidate for the treatment of inflammatory diseases.

The available compositions may not be effective in reducing the viral infection as they do not match the epidemic virus type and thus, there is a need to develop a viral therapeutic agent that is effective in preventing infection with high stability. Hence, there is a need for a formulation, which is effective against coronavirus.

### SUMMARY

The present disclosure relates to in silico (or in-silico) analysis to examine the natural-based compounds as inhibitors of Severe Acute Respiratory Syndrome Corona Virus 2 (SARS-CoV-2). The analysis identified Kobophenol A as a suitable inhibitor of SARS-CoV-2 based on the interaction between the human Angiotensin-Converting Enzyme 2 (ACE2) receptor and Spike receptor-binding domain (S1-RBD) of SARS-CoV-2 for treatment of infection caused by Corona Virus Disease-2019 (Covid-19).

The in-silico analysis is performed using the natural-based compounds as potential inhibitors of SARS-CoV-2, The in-silico analysis revealed that Kobophenol A effectively binds to the protein at two positions, namely ACE2/Spike interface and at hydrophobic pocket of the ACE2 domain through hydrogen bonds with a good docking energy. In addition to Kobophenol A, the metabolites M1, M2, and M3 of Kobophenol A also exhibited binding to bind at the ACE2/Spike interface and ACE2 hydrophobic pocket and accordingly Kobophenol A is considered for antiviral studies.

The inhibitory activity of Kobophenol A in vitro is analyzed using ELISA. The results of the in vitro inhibitory activity of Kobophenol A indicated that the increasing concentrations of Kobophenol A is effective in inhibiting ACE2 binding to SARS-CoV-2 S1-RBD with an IC₅₀ of 1.81±0.04 μM. The inhibitory activity is further validated by phenotypic virus-cell based antiviral assay in in VeroE6-EGFP cells. The results indicated that the cells treated with Kobophenol A inhibited the cell death caused by viral infection without inducing cell toxicity in the absence of viral infection.

The present disclosure also discloses the molecular analysis for confirmation and analysis of the stability and confirmation changes of the binding of Kobophenol A. The molecular dynamics is performed using Root-mean-square deviations (RMSD) and Root-Mean-Square Fluctuations (RMSF) of the backbone protein atoms within the ACE2 and S1-RBD binding regions. The RMSD plot indicated that when Kobophenol A is bound at the ACE2/Spike interface, the S1-RBD region rapidly equilibrated, whereas the ACE2 receptor required ˜200 ns to stabilize. Similarly, when Kobophenol A is instead bound in the hydrophobic pocket of the ACE2 domain, the S1-RBD region again quickly equilibrated but the ACE-2 receptor took a more substantial time of ˜350 ns to stabilize as illustrated in FIG. 4. The calculations from the RMSD suggest that more significant conformational changes occur within the ACE2 region relative to the S1-RBD regardless to the binding site, the ACE2/Spike interface or the ACE2 hydrophobic pocket. The RMSF analysis suggests that the binding location of Kobolphenol A has a bigger effect on the structural conformation of the S1-RBD region, whereas a much smaller conformational difference is observed for ACE2 receptor region.

The present disclosure disclosed that the crystal structure of the SARS-CoV-2 spike receptor-binding domain bound to the ACE2 receptor involves 17 hydrogen bonds and two salt bridges occurring between S1-RBD and ACE2. The electrostatic interactions reported between the S1-RBD and ACE2 receptors includes N487-Q24, K417-D30, Q493-E35, Q493-E37, Y505-E37, Y505-D38,Y449-D38, T500-Y41, N501-Y41, G446-Q42, Y449-Q42, Y489-Y83, N487-Y83, N487-Q325, N487-E329, N487-N330, G502-K353, Y505-R393 and 1(417-D30. It is also observed that five hydrogen bonds, namely N487-Q24, Q493-E35, Y449-D38, N487-Y83, and G502-K353, remained intact and regardless of binding of Kobophenol A.

The present disclosure also discloses that the binding affinity of Kobophenol A to the ACE2/Spike interface region and the ACE2 hydrophobic pocket as −19.0±4.3 and −24.9±6.9 kcal/mol, respectively.

The disclosure discloses Kobophenol A as a potential active ingredient for treatment of infection caused by SARS-CoV-2. The in-silico analysis reveals that Kobophenol A has shown high free binding affinity especially with spike (S) protein, and the additional anti-inflammatory, bronchodilator, cardioprotective and antioxidant activities of Kobophenol A is a promising health benefit for COVID-19 patients and may potentially help to reduce the mortality in the Covid-19 patients.

### BRIEF DESCRIPTION OF DRAWINGS

FIG. 1 tabulates the list of natural compounds identified using in silico analysis.

FIG. 2 illustrates the dose-dependent inhibition curve of soluble hACE2 binding to SARS-CoV-2 RBD in the presence of Kobophenol A.

FIGS. 3A and 3B illustrate the effect of Kobophenol A cell based antiviral assay with SARS CoV2.

FIG. 4 illustrates the RMSD analysis of Kobophenol A binding.

FIG. 5 illustrates the RMSF analysis of Kobophenol A binding.

FIG. 6 provides the details of the hydrogen bond percent occupancy.

FIG. 7 illustrates the computational analysis of new hydrogen bonds between ACE2 and the S1-RBD receptor.

FIG. 8 illustrates the interaction of ACE2 and S1-RBD at Y495-K353 using the molecular dynamics.

FIG. 9 illustrates the distance analysis of ACE2 and S1-RBD at Y495 and K353.

FIG. 10 tabulates the details of free energy of binding of Kobophenol A.

### DETAILED DESCRIPTION

In order to more clearly and concisely describe and point out the subject matter of the claimed disclosure, the following definitions are provided for specific terms, which are used in the following written description.

The term “Carrier” or “Pharmaceutical carrier” refers to diluents, adjuvants, excipients, or vehicles with which a compound of the disclosure is administered.

The term “Isomer” refers to any stereoisomer, enantiomer or diastereomer of any compound of the disclosure.

The term “Pharmaceutically acceptable” means approved by a regulatory agency of the Federal or a State Government or listed in the U.S. Pharmacopeia or other generally recognized pharmacopeia for use in animals and more particularly in humans.

The term “Stereometrically pure” means a composition that comprises one stereoisomer of a compound and is substantially free of other stereoisomers of that compound.

The term “In-Silico Analysis” refers to analysis of the activity of individual compound to analyze the interaction of compound with proteins using a computer simulation.

The present disclosure discloses in silico drug design analysis to examine the natural-based compounds as inhibitors of SARS-CoV-2. The analysis includes the identification of 25 compounds including some metabolites with the docking energies.

The spike (S) protein of coronavirus is a type I transmembrane envelope glycoprotein, which consists of S1 and S2 domains responsible for virus binding and fusion, respectively. The S1 domain contains a receptor-binding domain (RBD) that specifically binds to angiotensin-converting enzyme 2 (ACE2), the receptor present on the target cells. The S2 domain facilitates the fusion of the viral membrane into the host cell membrane. The S proteins of coronaviruses are responsible for virus binding, fusion and entry and are major inducers of neutralizing antibodies in addition to critical role in viral pathogenesis and spread of virulence. Hence, the spike proteins are crucial for the viral life cycle, and it is believed to serve as a major target to block the viral entry into the host cells.

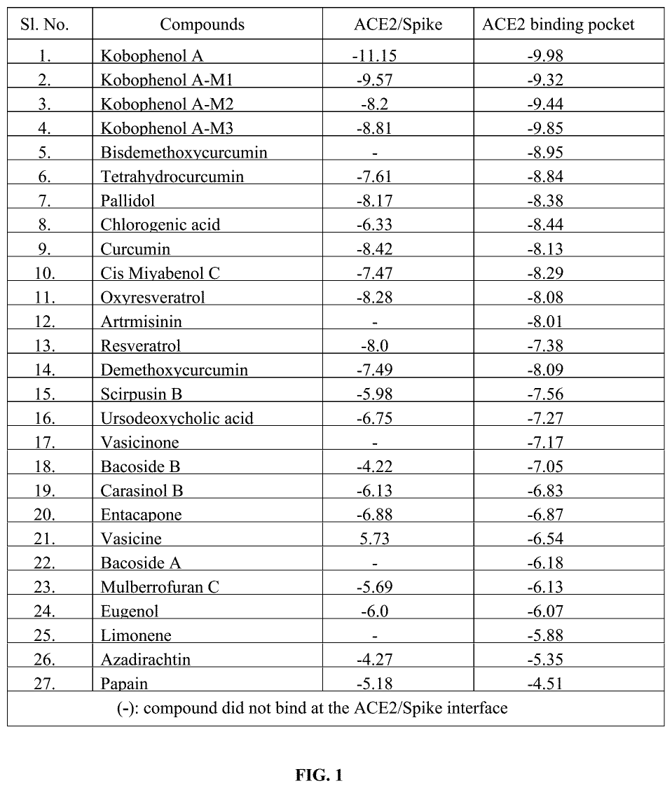

FIG. 1 tabulates the list of natural compounds identified using in silico analysis. The in silico analysis revealed that Kobophenol A effectively binds to the protein. The protein binding is achieved at two positions, firstly at ACE2/Spike interface through a hydrogen bond with residue Gln325 with a docking energy of −11.15 kcal/mol and secondly at the hydrophobic pocket of the ACE2 domain through hydrogen bonds with Glu375 and Thr347 with a docking energy of −9.98 kcal/mol.

These in silico-predicted interactions inhibit the binding of the SARS-CoV-2 spike protein with host ACE2 by destabilizing the complex formation. Additionally, three metabolites of Kobophenol A, namely M1, M2, and M3, also found to bind at the ACE2/Spike interface and ACE2 hydrophobic pocket with relatively high favorable docking energies in comparison to the other natural compounds in the library. Hence, Kobophenol A is considered for further in vitro studies and molecular mechanics targeting the ACE2/Spike RBD binding domains.

Kobophenol A is analyzed for in vitro ACE2/S1-RBD inhibitory activity in further studies to confirm the inhibitory activity of Kobophenol A. The inhibitory activity of Kobophenol A is analyzed by using ELISA. The assay is initiated by using a 96-well plate coated with recombinant 2019-nCoV S1-RBD at 0.1 to 0.4 μg/ml overnight. The plates are washed 3× with PBS pH 7.2 (without Ca2+ and Mg2+) with 0.05% Tween-20 and block with 1% BSA in PBS. 0.1 to 0.2 μg/ml of ACE2 receptor protein is added in the presence or absence of Kobophenol A at various concentrations such as 0.01 μM, 0.1 μM, 1 μM, 10 μM and 100 μM. The samples are incubated for 1-2 hours in the binding buffer comprising 0.1% BSA in PBS, pH 7.2. Finally, the plates are washed and anti-Human Fc-antibody-HRP 1:20,000 in binding buffer is added. After three washes 3,3′,5,5′- Tetramethylbenzidine (TMB) is added for signal, after stopping the reaction with an acidic solution the plates are read at 450 nm and IC50 is calculated.

FIG. 2 illustrates the dose-dependent inhibition curve of soluble hACE2 binding to SARS-CoV-2 RBD in the presence of Kobophenol A. The results of the in vitro inhibitory activity of Kobophenol A indicated that the increasing concentrations of Kobophenol A is effective in inhibiting ACE2 binding to SARS-CoV-2 S1-RBD with an IC50 of 1.81±0.04 μM. This inhibition suggests that Kobophenol A may inhibit the viral entry into the host and serve as a lead compound for anti-SARS-CoV-2 treatment.

The in vitro inhibition of binding of hACE2 to SARS-CoV-2 RBD is further validated by a phenotypic virus-cell based antiviral assay. A phenotypic virus-cell based antiviral assay of Kobophenol A is performed against SARS-CoV-2 in VeroE6-EGFP cells.

VeroE6-EGFP cells are propagated in a growth medium prepared by supplementing DME-M (Gibco Cat. No: 41965-039) with 10% v/v heat-inactivated FCS and 5 mL sodium bicarbonate 7.5%. The cells are cultured in T150 bottle and split ¼ twice a week. Pen-strep is added directly to the T150 bottle at a 1/100 dilution. The assay medium is prepared by supplementing DMEM with 2% v/v heat-inactivated FCS and 5 mL sodium bicarbonate 7.5%. 100 μL of medium is added to columns 1-12 of Greiner Bio One 655090 plate. 100 μL medium is added to column 12. 50 μL of medium is added to columns 11 and 2. 50 μL medium is added to column 2 Kobophenol A is added to column 2, rows B-G and further diluted over the plate.

A T150 cell culture flask containing a confluent cell monolayer is washed with DPBS, after which 10 mL Trypsin/EDTA is added. The trypsin is left on the cells for 1 minute, ascertaining the full monolayer has been in contact by gently tilting the cell culture flask. A volume of 8 mL of the liquid is removed, leaving 2 mL on the cell monolayer. The cell culture is incubated for 15 minutes at 37° C., after which the cells are resuspended in 10 mL of assay medium comprising DMEM with 2% FCS and 5 mL sodium bicarbonate without addition of penicillin or streptomycin. The cell suspension is passed through a cell Stainer to remove cell clumps. The amount of harvested cells is quantified by analyzing 3 samples of 10 μL cell suspension in 10 mL of isotonic buffer using a Coulter Counter. A cell suspension with a density of 25000 cells/50 μL is prepared in assay medium. 50 μl of this cell suspension is seeded to each well of the plate and the plates are incubated overnight at 37° C. with 5% CO₂.

The addition of virus to the assay is achieved by preparing virus to appropriate dilution in assay medium. SARS2 stock SARS2_Belgium_20200414 is used to prepare 1/50,000 dilution with a final dilution in the plate is 200,000, which has a titer of 2×10⁷ TCID50/mL. The final titer in the experiment is therefore 100 TCID50/mL=20 TCID50/well. With 25000 cells/well the MOI=0.001 TCID50/cell. 50 μL of this virus preparation is added to columns 1-10. The plates are incubated at 37° C. and 5% CO₂. On day 4, the plates are transferred to a high-content imager for determination of the GFP signal using high-content imaging. The number of fluorescent pixels above threshold is used as the read-out. The percentage inhibition is calculated by subtracting the background (number of fluorescent pixels in untreated/infected control wells) and normalizing to control wells without virus (also background subtracted). The cytotoxicity assay is identical to the antiviral assay with the difference that assay medium without virus is added instead of assay medium with virus.

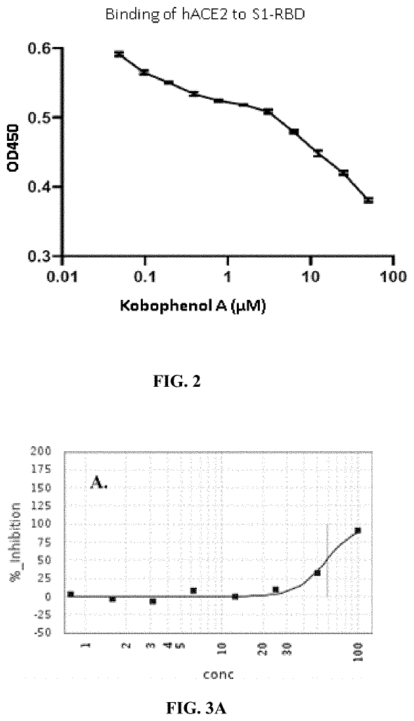

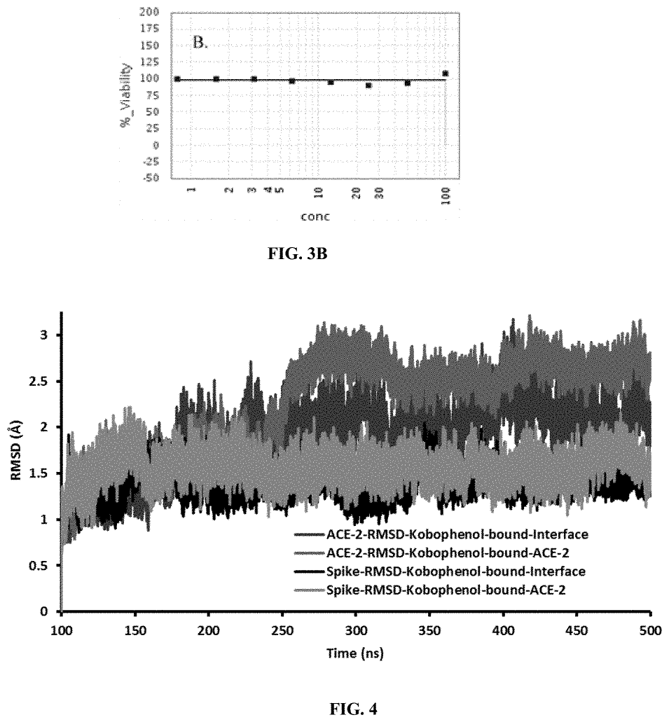

FIGS. 3A and 3B illustrate the effect of Kobophenol A cell based antiviral assay with SARS CoV2. The results indicated that the cells infected with virus with or without treatment of Kobophenol A showed an increase in VeroE6 signal, yielded a 50% maximum effective concentration (EC₅₀) value of 71.6 μM. This EC₅₀ is similar to values of known FDA approved drugs such as Indinavir (EC₅₀=59.14 μM), Favipiravir (EC₅₀=61.88 μM) and better than Penciclovir (EC₅₀=95.96 μM) and Ribavirin (EC₅₀=109.50 μM). Both the IC₅₀ value of Kobophenol A against recombinant 2019-nCOV Spike (RBD)/hFc protein and EC₅₀ value in VeroE6-EGFP cells fit the computational predictions that Kobophenol A inhibits the binding of S1-RBD of SARS-CoV-2 to the host ACE2 receptor. FIG. 3A illustrates that the increasing concentrations of Kobophenol A inhibited VeroE6-EGFP cells after four days and FIG. 3B indicates that the cells treated with Kobophenol A inhibited the cell death caused by viral infection without any cell toxicity in the absence of viral infection and is performed to monitor the toxicity of the compound cell viability using MTS assay.

According to an embodiment of the disclosure, the molecular dynamics is analyzed to determine any interactions or conformational changes arising from binding of Kobophenol A into the two potential sites namely ACE2/Spike interface or ACE2 hydrophobic pocket. The crystal structure of SARS-CoV-2 is retrieved from the rcsb.org (PDB ID: 6M0J)6 and used to generate initial 3-Dimensional (3D) coordinates of the Spike S1 -RBD-ACE2 complex. The co-crystallized water molecules are deleted, and the polar hydrogen molecules are added and Gasteiger charges are computed. The structures of selected natural compounds are superimposed against the pre-docked ligand in the PDB, and the latter is then removed to generate initial conformation of natural compound at the active site of SARS-CoV-2. As the natural compounds are not available in the x-ray crystal structure of S1-RBD bound with ACE2, a grid box is generated by considering the whole protein and blind docking is performed (PDB ID: 6M0J)6.

Finally, both the Autogrid and AutoDock are run with the default parameters and the top scoring molecules are evaluated for their interactions.

In the present disclosure, the molecular dynamics is achieved using Root-mean-square deviations (RMSD) and Root-Mean-Square Fluctuations (RMSF) of the backbone protein atoms within the ACE2 and S1-RBD binding regions.

FIG. 4 illustrates the RMSD analysis of Kobophenol A binding. The RMSD calculations provide a sense of the timescale required to stabilize the protein structure after substrate binding. Accordingly, the RMSD plot is divided into two parts namely the ACE2 receptor at residues 19-615 and the SARS-CoV-2 S1-RBD at residues 333-526. The RMSD plot indicated that when Kobophenol A is bound at the ACE2/Spike interface, the S1-RBD region rapidly equilibrated, whereas the ACE2 receptor required ˜200 ns to stabilize. Similarly, when Kobophenol A is instead bound in the hydrophobic pocket of the ACE2 domain, the S1-RBD region again quickly equilibrated but the ACE-2 receptor took a more substantial time of ˜350 ns to stabilize as illustrated in FIG. 4. The calculations from the RMSD suggest that more significant conformational changes occur within the ACE2 region relative to the S1-RBD regardless to the binding site, the ACE2/Spike interface or the ACE2 hydrophobic pocket.

The significance of binding of Kobophenol A is further explored using a RMSF analysis. RMSF analysis provides the positional deviations over time relative to a reference structure.

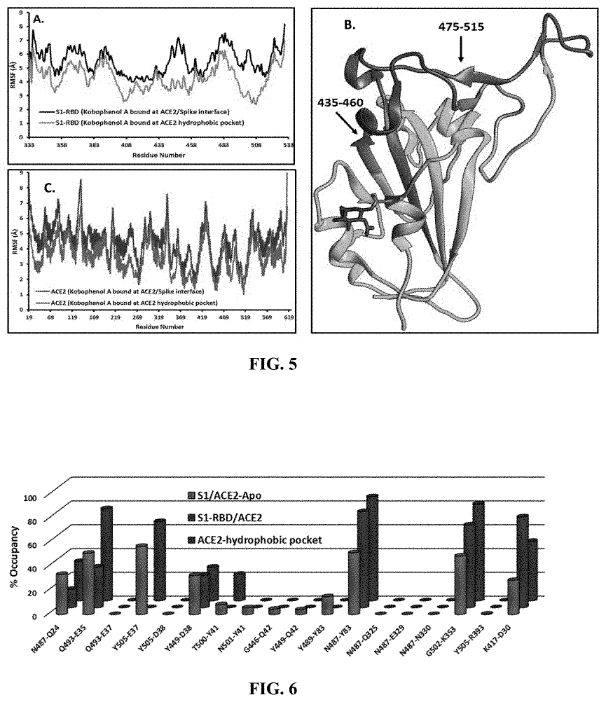

FIG. 5 illustrates the RMSF analysis of Kobophenol A binding. The RMSF analysis for the S1-RBD region of the protein at residues 333-533 is simulated upon binding of Kobophenol A at both proposed binding sites and greater fluctuations are computed when Kobophenol A is located at the ACE2/Spike interface as compared to the ACE2 hydrophobic pocket as depicted in FIG. 5A. The binding of Kobophenol A at the ACE2/Spike interface produced large fluctuations, particularly within residues ranging from 435 to 460 and 475 to 515, which constitutes the receptor binding motif (RBM) of S1 in the system as depicted in FIG. 5B. The residues located in the ACE2 domain are computed to have similar fluctuations for both binding motifs, although binding of Kobophenol A at the ACE2/Spike interface, which provided larger and absolute fluctuation distances compared to binding within the ACE2 hydrophobic pocket as in FIG. 5C. Accordingly, the RMSF analysis suggests that the binding location of Kobolphenol A has a bigger effect on the structural conformation of the S1-RBD region, whereas a much smaller conformational difference is observed for ACE2 receptor region.

It is observed that the crystal structure of the SARS-CoV-2 spike receptor-binding domain bound to the ACE2 receptor involves 17 hydrogen bonds and two salt bridges occurring between S1-RBD and ACE2. The electrostatic interactions reported between the S1-RBD and ACE2 receptors includes N487-Q24, K417-D30, Q493-E35, Q493-E37, Y505-E37, Y505-D38, Y449-D38, T500-Y41, N501-Y41, G446-Q42, Y449-Q42, Y489-Y83, N487-Y83, N487-Q325, N487-E329, N487-N330, G502-K353, Y505-R393, and K417-D30. These intermolecular interactions are monitored in the molecular dynamics simulations to estimate the extent to which these favorable interactions have been altered as a response to Kobophenol A binding in comparison to the unbound namely Apo System.

FIG. 6 provides the details of the hydrogen bond percent occupancy. It is to be noted that the differences in computed hydrogen bonding interactions for the Apo system and the systems with Kobophenol A bound at either pocket are negligible. Interestingly, over half of the hydrogen bond interactions that are present in the crystal structure are eliminated for all three systems. This suggests that a significant number of the favorable electrostatic interactions present in the crystal structure are not crucial for binding of the ACE2 receptor to the S1-RBD and is attributed to the conditions of crystallization. Instead, the residues are forming hydrogen bonds with other residues located within the individual proteins themselves, i.e., S1- RBD-to-S1-RBD residues or ACE2-to-ACE2 residues. It also to be to be observed that out of the original crystal structure hydrogen bonds identified between the two proteins, five hydrogen bonds namely N487-Q24, Q493-E35, Y449-D38, N487-Y83, and G502-K353 remained intact and regardless of binding of Kobophenol A. In addition, two more hydrogen bonds interactions, i.e., Y505-E37 and T500-Y41, are preserved when Kobophenol A is bound solely in the ACE2 hydrophobic pocket. A salt bridge reported between K417-D30 also remained regardless of substrate binding location.

As a result of molecular dynamics analysis, a new set of interactions and hydrogen bonds are computationally identified between ACE2 and the S1-RBD receptor.

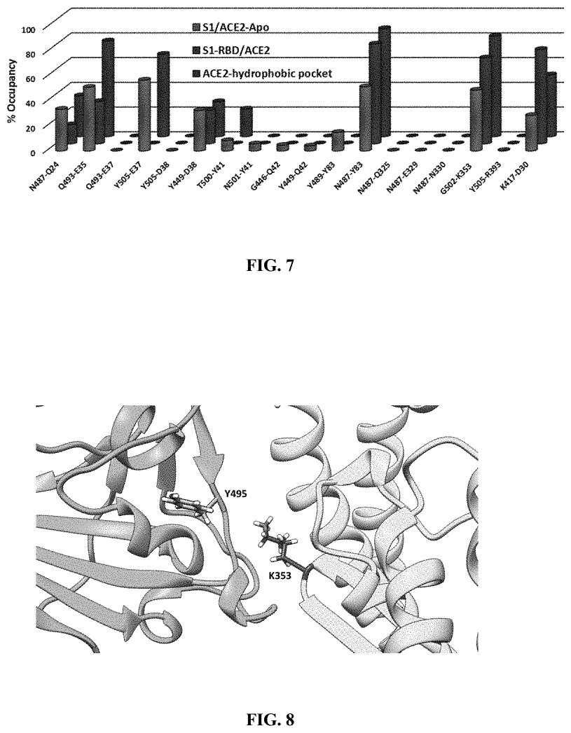

FIG. 7 illustrates the computational analysis of new hydrogen bonds between ACE2 and the S1-RBD receptor. The hydrogen bonds namely T500-D355, G502-D355, Y495-K353, and Q493-K31 are observed in crystal structure as depicted in the figure. It is observed that out of these interactions, the Y495-K353 hydrogen bond between the hydroxy group of Y495 in S1-RBD and the nitrogen atom of K353 in ACE2 domain is particularly interesting as it is observed in Apo simulation for 57% of time but eliminated when the Kobophenol A was bound in either pocket.

FIG. 8 illustrates the interaction of ACE2 and S1-RBD at Y495-K353 using the molecular dynamics. The molecular dynamics interpreted that the Y495-K353 interaction is located at the core center region of the interface-pocket formed between ACE2 and S1-RBD and aid to stabilize the interaction between both domains.

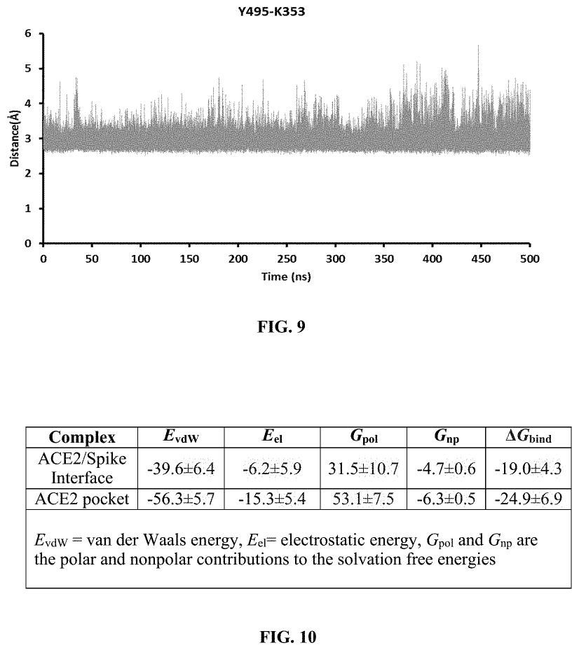

FIG. 9 illustrates the distance analysis of ACE2 and S1-RBD at Y495 and K353. The molecular dynamics interpreted that the distance analysis over the entire molecular dynamics trajectory of the Apo system found the O—H . . . NH2 interaction between Y495 and K353 maintained an average distance of 2.95 Å and its elimination upon ligand binding suggest the origin of inhibition.

According to an embodiment of the disclosure, the free energy of binding of Kobophenol A is estimated using molecular dynamic simulations.

FIG. 10 tabulates the details of free energy of binding of Kobophenol A. The analysis of free energy upon binding of Kobophenol A is estimated using the molecular dynamic simulations. The free energy of binding of Kobophenol A is achieved through the combination of molecular mechanics energies with the Poisson-Boltzmann surface area continuum solvation (MM/PBSA) method. The binding affinity of Kobophenol A to the ACE2/Spike interface region and the ACE2 hydrophobic pocket is computed to be −19.0±4.3 and −24.9±6.9 kcal/mol, respectively, over the course of the 500 ns trajectory as depicted in the FIG. 10. In order to understand the substantial preference for Kobophenol A in the ACE2 hydrophobic pocket, the individual energy contributions to the binding affinity are examined and the results interpreted that the van der Waals energy contribution (EvdW) and the polar contribution to the solvation free energies (Gpol) nearly cancel themselves out suggesting that the electrostatic energy contribution of the ACE2 hydrophobic pocket that is more than double of that of the ACE2/Spike interface, i.e., −15.3 versus −6.2 kcal/mol and is considered as a major contributor to the ACE2 pocket preference in the net binding free energy calculation.

The disclosure discloses Kobophenol A as a potential active ingredient for treatment of infection caused by SARS-CoV-2. The disclosure discloses an identification of free binding affinity of Kobophenol A to spike proteins and) (M^{pro}) Main protease of SARS-Co-V-2. This is followed by analysis of antiviral activity of Kobophenol A.

The present disclosure discloses the potential of Kobophenol A as an active ingredient along with other excipients. The in-silico analysis of Kobophenol A is achieved using X-ray crystal structure of spike protein and Main protease. The in-silico analysis reveals that Kobophenol A has shown high free binding affinity especially with spike (S) protein and M protease. The additional anti-inflammatory, bronchodilator, cardioprotective and antioxidant activities of Kobophenol A is a promising health benefit for COVID-19 patients and may potentially help to reduce the mortality in the Covid-19 patients.

According to an embodiment of the disclosure, the active ingredient is basic in nature to which it is not restricted but is capable of forming a wide variety of salts with various inorganic and organic acids. The acids that may be used to prepare pharmaceutically acceptable acid salts of such compounds are those that form non-toxic acid addition salts, i.e., salts containing pharmacologically acceptable anions, including but not limited to sulfuric, acetic, nitrate, hydrochloride, hydrobromide, acetate, phosphate, citric, oxalic, hydroiodide, maleic, sulfate, bisulfate, acid phosphate, isonicotinate, lactate, salicylate, citrate, acid citrate, tartrate, tannate, pantothenate, bitartrate, ascorbate, succinate, maleate, gentisinate, fumarate, gluconate, glucoronate, saccharate, formate, benzoate, glutamate, methasulfonate, ethanesulfonte, benzenesulfonate, p-toluenesulfonate, mesylate, hydroxymethylsulfonate, and pamoate salts. Similarly, compounds of the disclosure that include ionizable hydrogens can be combined with different inorganic and organic bases to form the respective salts.

The formulation of Kobophenol A is prepared as tablet, capsule, suspension, semi-solid, solution, etc which is useful for oral delivery or in the form of injection.

The formulation of Kobophenol A may be safe without inducing any adverse effects and effective against activation of coronaviruses as well as other viruses due to specific activity of Kobophenol A. The herbal supplements alone or in combination with other ingredients may result in synergistic action in treating COVID-19 patients.

The analysis of the present disclosure suggests that the natural based, oligomeric stilbenoid Kobophenol A from Caragana sinica effectively suppressed the interaction between the ACE2 receptor and S1-RBD domain of SARS-Co-V-2 with a vitro IC₅₀ value of 1.81 μM for Kobophenol A against recombinant 2019-nCOV Spike (RBD)/hFc protein and a an EC5( )value of 71.6 μM from a phenotypic virus-cell based antiviral assay with SARS-CoV-2 in VeroE6 cells. Moreover, Kobophenol A did not induce any cytotoxicity with a CC₅₀ value of more than 100 μM.

The molecular dynamic simulation employed in the present disclosure interpreted that binding the substrate in either pocket eliminated a central core interaction, Y495-K353, found between the ACE2 and S1-RBD interface pocket. Further, the computed free energies of binding for Kobophenol A at the Spike/ACE2 interface and the ACE2 hydrophobic pocket using MM/PBSA calculations yielded values of −19.0±4.3 and −24.9±6.9 kcal/mol, respectively.

The electrostatic energy contribution of the ACE2 hydrophobic pocket is more than double of that of the ACE2/Spike interface when binding Kobophenol A, which confirms the preference.

Kobophenol A is computationally identified as a good lead compound effective against SARS-CoV-2 infection, which is further validated experimentally to inhibit the binding of S1-RBD from SARS-CoV-2 to the host ACE2 receptor. The obtained results suggested that Kobophenol A shall be further developed as a safe and effective drug without toxicity for SARS-CoV-2 infection.

1. A method for in-silico analysis of natural-based compounds as inhibitor of Severe Acute Respiratory Syndrome Corona Virus 2 (SARS-CoV-2), the method comprising:
a. screening at least 25 natural-based compounds;
b. analyzing the interaction between human angiotensin-converting enzyme 2 (ACE2) receptor and Spike receptor-binding domain (S1-RBD) of SARS-CoV-2 using a computer simulation; and
c. identifying a compound binding to SARS-CoV-2 protein, wherein the binding is achieved in at least two positions including ACE2/Spike interface and ACE2 hydrophobic pocket with relatively high favorable docking energies and the compound identified is Kobophenol A.

2. The method of claim 1, wherein Kobophenol A binds at ACE2/Spike interface through a hydrogen bond with residue Gln325 with a docking energy of −11.15 kcal/mol and at ACE2 hydrophobic pocket through hydrogen bonds with Glu375 and Thr347 with a docking energy of −9.98 kcal/mol.

3. The method of claim 1, wherein Kobophenol A blocked the interaction between the ACE2 receptor and S1-RBD in vitro with an IC50 of 1.81 ±0.04 μM and inhibited SARS-CoV-2 viral infection with an EC₅₀ of 71.6 μM in cells.

4. The method of claim 1, wherein Kobophenol A inhibited cell viability in VeroE6-EGFP cells infected with SARS-CoV-2.

5. The method of claim 1, wherein binding of Kobophenol A with SARS-CoV-2 as analyzed by molecular dynamics indicated the presence of at least 17 hydrogen bonds and at least two salt bridges occurring between S1-RBD and ACE2.

6. The method of claim 1, wherein binding of S1-RBD and ACE2 receptors comprises N487-Q24, K417-D30, Q493-E35, Q493-E37, Y505-E37, Y505-D38, Y449-D38, T500-Y41, N501-Y41, G446-Q42, Y449-Q42, Y489-Y83, N487-Y83, N487-Q325, N487-E329, N487-N330, G502-K353, Y505-R393, and K417-D30 electrostatic interactions.

7. The method of claim 1, wherein binding of S1-RBD and ACE2 receptors is stabilized at Y495 and K353 maintained an average distance of 2.95 Å.

8. The method of claim 1, wherein a binding affinity of Kobophenol A to the ACE2/Spike interface region is −19.0±4.3 kcal/mol and the at ACE2 hydrophobic pocket is −24.9±6.9 kcal/mol.

9. The method of claim 1, wherein Kobophenol A is considered as a potential drug for inhibition of SARS-CoV-2 infection and treatment of Corona Virus Disease-2019 (Covid-19).
